(19) United States
(12) Patent Application Publication
(10) Pub. No.: US 2005/0130165 A1
(43) Pub. Date: Jun. 16, 2005

(54) Polymorphism of soati useful for identifying risk of developing alzheimer's disease

(76) Inventors: Andreas Papasotiropoulos, Zurich (CH); Axel Wollmer, Zurich (CH); Roger Nitsch, Zollikon (CH); Christoph Hock, Erlenbach (CH)

Correspondence Address: JACOBSON HOLMAN PLLC, 400 SEVENTH STREET N.W., SUITE 600, WASHINGTON, DC, 20004, US

(21) Appl. No.: 10/514,823

(22) PCT Filed: May 16, 2003
(86) PCT No.: PCT/EP03/05190

Related U.S. Application Data
(60) Provisional application No. 60/380,932, filed on May 17, 2002.

(30) Foreign Application Priority Data
May 17, 2002 (EP) 02011029.2

(51) Int. Cl. C12Q 1/68

(52) U.S. Cl. 435/6

(57) ABSTRACT

Based on the unexpected association of a SOAT1 gene polymorphism with reduced genetic risk for a neurodegenerative disease, in particular Alzheimer's disease, the present invention provides a method of diagnosing or prognosticating such a disease, or determining the propensity or predisposition of a subject to develop such a disease. The method comprises detecting the presence or absence of a single nucleotide polymorphism in the SOAT1 gene which encodes the enzmye ACAT1, acyl-coenzyme A: cholesterol acyltransferase 1.

The present invention relates to methods of diagnosing, prognosticating and monitoring neurodegenerative diseases in a subject, based on the identification of the genetic association of SOAT1 gene polymorphisms with reduced genetic risk for a neurodegenerative disease, in particular Alzheimer's disease.

Neurodegenerative diseases, in particular Alzheimer's disease, have a severely debilitating impact on a patient's life. Furthermore, these diseases constitute an enormous health, social, and economic burden. Alzheimer's disease is the most common age-related neurodegenerative condition affecting about 10% of the population over 65 years of age and up to 45% over age 85 (for a recent review see Vickers et al., Progress in Neurobiology 2000, 60:139-165). Presently, this amounts to an estimated 12 million cases in the US, Europe, and Japan. This situation will inevitably worsen with the demographic increase in the number of elderly persons (“aging of the baby boomers”) in developed countries. The neuropathological hallmarks that occur in the brain of individuals suffering from Alzheimer's disease are senile plaques, composed of amyloid-β protein, and profound cytoskeletal changes coinciding with the appearance of abnormal filamentous structures and the formation of neurofibrillary tangles.

AD is a progressive disease that is associated with early deficits in memory formation and ultimately leads to the complete erosion of higher cognitive function. Alzheimer's disease is genetically complex. The risk for the development of AD is determined by variations of genes involved in major pathophysiological pathways of this disorder. A considerable part of this risk is attributed to the inheritance of the e4 allele of the apolipoprotein E gene (APOE*4). However, several additional genes and genetic interactions add to the overall genetically determined susceptibility for the development of AD. Genes coding for proteins involved in central disease-related pathways are of particular interest in the genetics of AD. The overproduction and aggregation of the β-amyloid peptide (Aβ) in the hippocampus and the medial lobe (MTL) is a crucial step in the pathogenesis of AD. Thus, genes implicated in mechanisms leading to Aβ accumulation are promising candidates in the search for susceptibility genes of AD.

Brain deposition of β-amyloid peptide (Aβ) is a crucial step in the pathogenesis of AD (Hardy. et al., Science 1992, 256: 184-5). It can cause the formation of neurofibrillary tangles within neurons (Gotz et al., Science 2001, 293: 1491-5; Lewis et al., Science 2001, 293: 1487-91). The concentration of the amyloid peptide Aβ42 may be used as a surrogate, quantitative trait to identify genetic loci for AD (Ertekin-Taner et al., Science 2000, 290: 2303-4). Thus, genes implicated in the regulation of Aβ formation and its degradation are candidate susceptibility genes for AD.

Intracellular and membrane-bound cholesterol is a regulator of the production of the β-amyloid peptide (Aβ), which is central to the pathogenesis of Alzheimer's disease (Selkoe, Physiol Rev 2001, 81: 741-66). High cellular cholesterol promotes and low cellular cholesterol reduces Aβ-production and release (Simons et al., Proc Natl Acad Sci USA 1998, 95: 6460-4; Fassbender et al., Proc Natl Acad Sci USA 2001, 98: 5856-61). However, the pathways involved in this regulation remain to be identified. Recently, Puglielli et al. (Nature Cell Biol 2001, 3: 905-912) contributed significantly to the understanding of the mechanisms implicated in the cholesterol-related Aβ-production. The equilibrium of cellular free cholesterol and cholesterol esters is under the tight control of acyl-coenzyme A: cholesterol acyltransferase 1 (ACAT1; Genbank protein identification: XD_{—}086193.1) which esterifies cholesterol with long chain fatty acids (Rudel et al., Curr Opin Lipidol 2001, 12: 121-127; Chang et al., Curr Opin Lipidol 2001, 12: 289-96; Chang et al., Annu Rev Biochem 1997, 66: 613-38). This esterification facilitates the transfer of cholesterol from the cell membrane to intracellular lipid droplets in the form of neutral cholesteryl esters. Furthermore, ACAT1 plays an important role in steroid hormone production, dietary cholesterol absorption, and lipoprotein assembly (Suckling et al., J. Lipid. Res. 1985, 26: 647-671). Functional deficiency or inhibition of ACAT1 drastically decreases Aβ production in vitro. Thus, intracellular compartmentation of cholesterol seems to be crucial for the metabolism of Aβ, and ACAT1 is involved in this process. SOAT1, the gene encoding ACAT1 maps to human chromosome 1q25, a broad chromosomal region associated with AD in full genome scans (Kehoe et al., Hum Mol Genet 1999, 8: 237-45; Myers et al., Am J Med Genet 2002, 114: 235-44; genomic sequence NCBI contig NT_{—}026949; Genbank mRNA accession number: XM_{—}086193, L21934). Thus, SOAT1 is a positional candidate gene with a probability of being a susceptibility gene for AD (Sullivan et al., Arch Gen Psychiatry 2001, 58: 1015-24).

ACAT1 is expressed in the adrenal gland, kidney, liver, lung, placenta, intestine, pancreas and brain (Yu et al, J. Biol. Chem. 1999, 274:36139-36145; Levy et al., FASEB J. 1995, 9:626-6356; Li et al., J. Biol. Chem. 1999, 274:11060-11071). In addition, ACAT1 is also expressed in the following cell types: macrophages, antigen-presenting cells, steroid hormone-producing cells, neurons, cardiomyocytes, smooth muscle cells, mesothelial cells, epithelial cells of the urinary tracts, thyroid follicles, renal tubules, pituitary, prostatic, and bronchial glands, alveolar and intestinal epithelial cells, pancreatic acinar cells, and hepatocytes (Sakashita et al., Am. J. Pathol. 2000, 156:227-236).

So far, 4 splice variants of 7 kb, 4.3 kb, 3.6 kb, and 2.8 kb have been reported. Interestingly, the 4.3kb-splice variant is hypothesized to be encoded on chromosome 1 and 7 and to be trans-spliced by a novel RNA recombination mechanism (Li et al., J. Biol. Chem. 1999, 274:11060-11071). The open reading frame comprises 1653 base pairs encoding a protein of 550 amino acids with a molecular weight of approximately 65 kDa (Genbank mRNA accession number: NP003092.1, Swiss-Prot. #P35610). The enzyme is located mainly in the membrane of the rough endoplasmic reticulum (Sakashita et al., Am. J. Pathol. 2000, 156:227-236) and comprises seven transmembrane helices (Lin et al., J. Biol. Chem. 1999, 274:23276-23285). ACAT1 is active as a tetrameric enzyme which is allosterically regulated by cholesterol (Yu et al., J. Biol. Chem. 1999, 274:36139-36145; Chang et al., J. Biol. Chem. 1998, 273:35132-35143). Disruption of the mouse ACAT1-gene resulted in a decreased cholesterol esterification mostly in fibroblasts and adrenal membranes but not in liver, suggesting the existence of additional esterification enzymes (Meiner et al., Proc. Natl. Acad. Sci. USA 1996, 93:14041-14046). Indeed, the human orthologue ACAT2 has been cloned recently which shares a 56%-identity with ACAT1 and in contrast to ACAT1 is mainly expressed in intestine and liver (Oelkers et al., J. Biol. Chem. 1998, 273:26765-26771). Both enzymes are regulated differentially by long chain free fatty acids in tissue culture cells, ACAT1 showing a strong preference for oleic acid (Seo et al., Biochemistry 2001, 40:4756-4762). Furthermore, the catalytic domains of ACAT1 and ACAT2 are likely to be oriented to opposite sides of the membrane (Joyce et al., Curr. Opin. Lipidol. 1999, 10:89-95). ACAT1 may be induced by 1,25-dihydroxyvitamin D(3) or cis-retinoic acid in THP1-cells (Maung et al., J. Lipid. Res. 2001, 42:181-187) and-ACAT1-mRNA is upregulated in mice fed with a high fat and cholesterol diet presumably due to free fatty acids and more unlikely due to cholesterol (Uelmen et al., J. Biol. Chem. 1995, 270:26192-26201). The mouse- and rat-homologues are approx. 85% identical to the human ACAT1. In addition, homologues have also been found in several organisms like yeast and drosophila.

ACAT1 has been implicated in the manifestation and progression of atheriosclerosis through the pathological accumulation of cholesterol esters. This has led to an intense search for inhibitors of ACAT1 in the pharmaceutical industry. Recently, a role for ACAT1 has been proposed in the generation of the amyloidogenic Abeta-peptide, which may play a major role in the development of Alzheimer's disease. The modulation of the intracellular cholesterol compartmentation by ACAT1 thereby affects Abeta generation, and inhibitors of ACAT1 which have been developed for the treatment of atheriosclerosis significantly lowered Abeta-generation (Puglielli et al., Nat. Cell Biol. 2001, 3:905-91).

It is crucial to expand the pool of potential drug targets and diagnostic markers. Therefore, it is an object of the present invention to provide methods of diagnosing or prognosticating a neurodegenerative disease, in particular Alzheimer's disease. A further objective of the present invention is to provide methods of monitoring the progression of such a disease and of evaluating a treatment for it. This objective is based on the identification of the genetic association of the SOAT1 gene, coding for ACAT1, with low cerebrospinal fluid levels of cholesterol, with low brain amyloid load, and with a reduced risk for Alzheimer's disease. The objective of the present invention has been solved by the methods and kits according to the features of the independent claims. Further preferred embodiments of the present invention are defined in the sub-claims thereto.

The singular forms “a”, “an”, and “the” as used herein and in the claims include plural reference unless the context dictates otherwise. For example, “a cell” means as well a plurality of cells, and so forth. The term “and/or” as used in the present specification and in the claims implies that the phrases before and after this term are to be considered either as alternatives or in combination. For instance, the wording “determination of a level and/or an activity” means that either only a level, or only an activity, or both a level and an activity are determined. The term “level” as used herein is meant to comprise a gage of, or a measure of the amount of, or a concentration of a transcription product, for instance an mRNA, or a translation product, for instance a protein or polypeptide. The term “activity” as used herein shall be understood as a measure for the ability of a transcription product or a translation product to produce a biological effect or a measure for a level of biologically active molecules. The term “activity” also refers to enzymatic activity. The terms “level” and/or “activity” as used herein further refer to gene expression levels or gene activity. Gene expression can be defined as the utilization of the information contained in a gene by transcription and translation leading to the production of a gene product. “Dysregulation” shall mean an upregulation or downregulation of gene expression. A gene product comprises either RNA or protein and is the result of expression of a gene. The amount of a gene product can be used to measure how active a gene is. The term “gene” as used in the present specification and in the claims comprises both coding regions (exons) as well as non-coding regions (e.g. non-coding regulatory elements such as promoters or enhancers, introns, leader and trailer sequences). The term “ORF” is an acronym for “open reading frame” and refers to a nucleic acid sequence that does not possess a stop codon in at least one reading frame and therefore can potentially be translated into a sequence of amino acids. “Regulatory elements” shall comprise inducible and non-inducible promoters, enhancers, operators, and other elements that drive and regulate gene expression. The term “fragment” as used herein is meant to comprise e.g. an alternatively spliced, or truncated, or otherwise cleaved transcription product or translation product. The term “derivative” as used herein refers to a mutant, or an RNA-edited, or a chemically modified, or otherwise altered transcription product, or to a mutant, or chemically modified, or otherwise altered translation product. For instance, a “derivative” may be generated by processes such as altered phosphorylation, or glycosylation, or acetylation, or lipidation, or by altered signal peptide cleavage or other-types of maturation cleavage. These processes may occur post-translationally. The term “modulator” as used in the present invention and in the claims refers to a molecule capable of changing or altering the level and/or the activity of a gene, or a transcription product of a gene, or a translation product of a gene. Preferably, a “modulator” is capable of changing or altering the biological activity of a transcription product or a translation product of a gene. Said modulation, for instance, may be an increase or a decrease in enzyme activity, a change in binding characteristics, or any other change or alteration in the biological, functional, or immunological properties of said translation product of a gene. The terms “agent”, “reagent”, or “compound” refer to any substance, chemical, composition or extract that have a positive or negative biological effect on a cell, tissue, body fluid, or within the context of any biological system, or any assay system examined. They can be agonists, antagonists, partial agonists or inverse agonists of a target. Such agents, reagents, or compounds may be nucleic acids, natural or synthetic peptides or protein complexes, or fusion proteins. They may also be antibodies, organic or anorganic molecules or compositions, small molecules, drugs and any combinations of any of said agents above. They may be used for testing, for diagnostic or for therapeutic purposes. The terms “oligonucleotide primer” or “primer” refer to short nucleic acid sequences which can anneal to a given target polynucleotide by hybridization of the complementary base pairs and can be extended by a polymerase. They may be chosen to be specific to a particular sequence or they may be randomly selected, e.g. they will prime all possible sequences in a mix. The length of primers used herein may vary from 10 nucleotides to 80 nucleotides. “Probes” are short nucleic acid sequences of the nucleic acid sequences described and disclosed herein or sequences complementary therewith. They may comprise full length sequences, or fragments, derivatives, isoforms, or variants of a given sequence. The identification of hybridization complexes between a “probe” and an assayed sample allows the detection of the presence of other similar sequences within that sample. As used herein, “homolog or homology” is a term used in the art to describe the relatedness of a nucleotide or peptide sequence to another nucleotide or peptide sequence, which is determined by the degree of identity and/or similarity between said sequences compared. The term “variant” as used herein refers to any polypeptide or protein, in reference to polypeptides and proteins disclosed in the present invention, in which one or more amino acids are added and/or substituted and/or deleted and/or inserted at the N-terminus, and/or the C-terminus, and/or within the native amino acid sequences of the native polypeptides or proteins of the present invention. Furthermore, the term “variant” shall include any shorter or longer version of a polypeptide or protein. “Variants” shall also comprise a sequence that has at least about 80% sequence identity, more preferably at least about 90% sequence identity, and most preferably at least about 95% sequence identity with the amino acid sequences of ACAT1. “Variants” of an ACAT1 protein molecule include, for example, proteins with conservative amino acid substitutions in highly conservative regions. “Proteins and polypeptides” of the present invention include variants, fragments and chemical derivatives of an ACAT1 protein. They can include proteins and polypeptides which can be isolated from nature or be produced by recombinant and/or synthetic means. Native proteins or polypeptides refer to naturally-occurring truncated or secreted forms, naturally occurring variant forms (e.g. splice-variants) and naturally occurring allelic variants. The term “isolated” as used herein is considered to refer to molecules that are removed from their natural environment, i.e. isolated from a cell or from a living organism in which they normally occur, and that are separated or essentially purified from the coexisting components with which they are found to be associated in nature. This notion further means that the sequences encoding such molecules can be linked by the hand of man to polynucleotides, to which they are not linked in their natural state, and that such molecules can be produced by recombinant and/or synthetic means. Even if for said purposes those sequences may be introduced into living or non-living organisms by methods known to those skilled in the art, and even if those sequences are still present in said organisms, they are still considered to be isolated.

The term “polymorphism” refers to the existence of more than one form of a gene or portion of a gene. It refers to a genetic variation in a nucleotide sequence at a given nucleotide position in the genome, within a given population, and a frequency usually exceeding 1%. Regions harboring polymorphisms may be a given gene region, coding or non-coding portions of the gene, or even intergenic regions, and are designated as “polymorphic regions”. They may cause differences in the nucleotide sequences as well as in the polypeptide sequences, in protein modifications, gene and protein expression processes and DNA replication. The term “single nucleotide polymorphism (SNP)” refers to a polymorphic variation in a nucleotide sequence at a given single nucleotide position in the genome. Single nucleotide polymorphisms may include any single base changes such as a deletion, insertion, or a base exchange. A single nucleotide polymorphism may cause a change in the encoded polypeptide sequence as well. A particular SNP may be indicative for a disease state, a specific feature, or for the risk of developing a disease. A “locus” of a gene refers to a unique position on a chromosome at which the genetic information lies and includes coding sequences, intervening sequences, and regulatory elements of the given gene. The distance between the loci of genes, the map distance, is expressed either in physical terms, or in genetic terms (recombination frequency). It is said that a gene maps to a specific locus on a chromosome. The locus at which the polymorphic variation occurs is the “polymorphic site or polymorphic marker”. The term “allele” or “allelic variant” refers to one of several alternative forms of a gene, or a portion thereof, typically having particular features which result in a particular phenotype. The term “allele” includes any inherited variation in the DNA sequence of a gene located at a given position in the genome. “Allele frequency” or “gene frequency” refers to the frequency with which a given allele is present at a given locus in a given population. It refers explicitly to the frequency of an allele in a population and not to the individual genotypes. The frequency distribution of two alleles in a population is following the formula (p+q)²=1.0, and the corresponding genotypes in the population are calculated according to p²+2pq+q²=1.0.

An individual or a subject is “homozygous” when two alleles of a given gene of a diploid organism are identical in respect to a given variation or polymorphism. By “heterozygous” is meant that the two alleles at a given locus are different. In the present invention, the terms “risk”, “susceptibility”, “propensity”, and “predisposition” are tantamount and are used with respect to the probability of developing a neurodegenerative disease, preferably Alzheimer's disease. A “haplotype” refers to the polymorphisms located on a single DNA strand, it refers to a series of alleles at several closely linked gene loci on a single chromosome. Thus “haplotyping” refers to the identification of polymorphisms on a single DNA strand. “Genotype”, is the genetic constitution of an individual or a cell, the types of alleles found at a given locus. “Linkage” in terms of genetics, refers to gene loci on the same chromosome, localized in a certain distance from each other, so that an independent segregation is not necessarily the case. The closer the gene loci lie to each other, the less frequently they are separated by recombination (crossing-over event between homologous chromosomes) and the higher the probability for linkage. Loci that are physically very close to each other, i.e. recombination between them will usually not occur, tend to be inherited together. The object of “linkage analysis” is to determine whether two loci tend to cosegregate more often than they would, if they were not physically close together. It is to estimate the recombination fraction, to test if the value is less than 0.5 (loci located on different chromosomes), and whether an observed deviation from 0.5 is statistically significant. The ratio of the probability that two gene loci are genetically linked, to the probability that they are not genetically linked, is referred to as “odds ratio”. If the odds for linkage exceed or reach a minimal value (ratio of 1000:1), it is assumed that two gene loci are linked. The “LOD score” (logarithm of the odds) refers to the logarithm of the calculated odds ratio, and is the likelihood of a given disease, or phenotype to be localized within the genetic region examined. The formula for determining the lod score is Z(θ)=log₁₀[L(θ)]−log₁₀[L(θ=0.5)]. “Linkage disequilibrium (LD)” refers to alleles which are nonrandomly associated at closely linked gene loci and operates over distances less than 1 cM. This association of alleles is not necessarily a consequence of close linkage. Allelic association will be shown only if the alleles mark conserved ancestral chromosomal segments, which is usually due to founder effects. The term “positional candidate gene” refers to a gene which is considered as a possible locus for a given disease. The assumption is based on known properties as function, expression pattern, chromosomal location etc. The term “susceptibility gene” refers to a gene locus which is associated with a given disease and which is a major risk factor for developing said given disease. A “genetic association study” is meant to be a test for differences in the distribution of alleles between unrelated affected subjects (patients diagnosed with a given disease) and controls (healthy subjects). It is tested whether a genetic variant (SNP of a gene) increases disease risk, i.e. is associated with a trait. LD may be also tested, whereby an increased prevalence of a characteristic set of SNP alleles in affected subjects will be identified.

The term ‘AD’ shall mean Alzheimer's disease. “AD-type neuropathology” as used herein refers to neuropathological, neurophysiological, histopathological and clinical hallmarks as described in the instant invention and as commonly known from state-of-the-art literature (see: lqbal, Swaab, Winblad and Wisniewski, Alzheimer's Disease and Related Disorders (Etiology, Pathogenesis and Therapeutics), Wiley & Sons, New York, Weinheim, Toronto, 1999; Scinto and Daffner, Early Diagnosis of Alzheimer's Disease, Humana Press, Totowa, N.J., 2000; Mayeux and Christen, Epidemiology of Alzheimer's Disease: From Gene to Prevention, Springer Press, Berlin, Heidelberg, N.Y., 1999; Younkin, Tanzi and Christen, Presenilins and Alzheimer's Disease, Springer Press, Berlin, Heidelberg, N.Y., 1998). Neurodegenerative diseases or disorders according to the present invention comprise Alzheimer's disease, Parkinson's disease, Huntington's disease, amyotrophic lateral sclerosis, Pick's disease, fronto-terniporal dementia, progressive nuclear palsy, corticobasal degeneration, cerebro-vascular dementia, multiple system atrophy, argyrophilic grain dementia and other tauopathies, and mild-cognitive impairment. Further conditions involving neurodegenerative processes are, for instance, age-related macular degeneration, narcolepsy, motor neuron diseases, prion diseases, traumatic nerve injury and repair, and multiple sclerosis.

In one aspect, the invention features a method for diagnosing or prognosticating a neurodegenerative disease in a subject, or determining the propensity or predisposition of a subject to develop a neurodegenerative disease. The method comprises detecting in a sample obtained from said subject the presence or absence of a variation in the SOAT1 gene, wherein the presence of a variation in the SOAT1 gene in said subject indicates a diagnosis or prognosis of a neurodegenerative disease, or a measure for the propensity or predisposition of developing a neurodegenerative disease as compared to a subject who does not carry a variation in said gene. A variation in the SOAT1 gene can be understood as any alteration in the naturally occuring nucleic acid sequence of the SOAT1 gene, i.e. any alteration from the wildtype.

In a preferred embodiment of the herein claimed methods of diagnosing, prognosticating and monitoring, of kits, recombinant animals, molecules, assays, and uses of the instant invention, said SOAT gene is the SOAT1 gene coding for the acyl-coenzyme A: cholesterol acyltransferase 1, also termed ACAT1, mapping to human chromosome 1q25 (Genbank protein identification number: XD_{—}086193.1; Genbank mRNA accession number: NP003092.1, XM_{—}086193, Swiss-Prot. number P35610). In the instant invention, said SOAT1 gene or ACAT1 protein is also generally referred to as the SOAT 1 gene or just SOAT1.

In a preferred embodiment, the variation in the SOAT1 gene is a single nucleotide polymorphism located at chromosomal position 180457672, 29 bp downstream from the 3′-end within the 3′ UTR of the ACAT1 mRNA, (single nucleotide polymorphism identification number: rs1044925). In a further preferred embodiment, the variation is a C to A transversion, i.e. the A-allele. In very close vicinity to said single nucleotide polymorphism there is a further variation in the SOAT1 gene, a single nucleotide polymorphism (chromosomal position 180457565, situated just within the 3′ UTR of the ACAT1 mRNA, single nucleotide polymorphism identification number: rs1803033). Due to this very close proximity of both polymorphisms it is likely that they are linked to each other. However, said further variation (rs1803033) may also act independently as a diagnostic polymorphism and may be used as a diagnostic marker according to the method of the present invention. In another preferred embodiment, further variations located within coding regions, intronic regions, within the 3′ UTR or the 3′ flanking region of the SOAT1 gene, in particular the SNPs rs3753526, rs3753527, rs4652366, rs2484437, rs2184575, rs2065761 and rs2065762, may also act independently as diagnostic polymorphisms and may be used as diagnostic markers according to the method of the present invention. In a further preferred embodiment, the presence of both copies of the A-allele, i.e. the A/A genotype, indicates a reduced propensity or predisposition of developing a neurodegenerative disease as compared to a subject who does not carry both copies of the A-allele.

In one further preferred embodiment of the herein claimed methods of diagnosing, prognosticating and monitoring, kits, recombinant animals, molecules, assays, and uses of the instant invention, said neurodegenerative disease or disorder is Alzheimer's disease, and said subjects suffer from Alzheimer's disease.

The method according to the present invention may be particularly useful for the identification of individuals that are at risk of developing a neurodegenerative disease. Consequently, the method, according to the present invention, may serve as a means for targeting identified individuals for early preventive measures or therapeutic intervention prior to disease onset, before irreversible damage in the course of the disease has been inflicted.

Determining the presence or absence of a polymorphism or variation in the SOAT1 gene may comprise determining a partial nucleotide sequence of the DNA from said subject, said partial nucleotide sequence indicating the presence or absence of said polymorphism or variation. It may further be preferred to perform a polymerase chain reaction with the DNA from said subject to determine the presence or absence of said polymorphism or variation. Such techniques are known to those skilled in the art (see Lewin B, Genes V, Oxford University Press, 1994).

The invention also relates to the construction and the use of primers and probes which are unique to the nucleic acid sequences of the SOAT1 gene, or fragments or variants thereof, as disclosed in the present invention. The oligonucleotide primers and/or probes can be labeled specifically with fluorescent, bioluminescent, magnetic, or radioactive substances. The invention further relates to the detection and the production of said nucleic acid sequences, or fragments and/or variants thereof, using said specific oligonucleotide primers in appropriate combinations. PCR-analysis, a method well known to those skilled in the art, can be performed with said primer combinations to amplify said gene specific nucleic acid sequences from a sample containing nucleic acids. Such sample may be derived either from healthy or diseased subjects. Whether an amplification results in a specific nucleic acid product or not, and whether a fragment of different length can be obtained or not, may be indicative for a neurodegenerative disease, in particular Alzheimer's disease. Thus, the invention provides nucleic acid sequences, oligonucleotide primers, and probes of at least 10 bases in length up to the entire coding and gene sequences, useful for the detection of gene mutations and single nucleotide polymorphisms in a given sample comprising nucleic acid sequences to be examined, which may be associated with neurodegenerative diseases, in particular Alzheimer's disease. This feature has utility for developing rapid DNA-based diagnostic tests, preferably also in the format of a kit.

In a preferred embodiment of the invention, the sample taken for genetic analysis comprises DNA obtained from body fluids, tissues, or any suitable cells of the body readily available. Preferably, the sample is a blood sample. However, the sample may also comprise body fluids such as saliva, urine, serum plasma, mucus, or cerebrospinal fluid.

In a, further aspect, the invention features a method for diagnosing or prognosticating a neurodegenerative disease in a subject, or determining the propensity or predisposition of a subject to develop a neurodegenerative disease, in particular AD, comprising determining a level, or an activity, or both said level and said activity, of at least one substance which is selected from the group consisting of a transcription product of the SOAT1 gene, and/or a translation product of the SOAT1 gene, and/or of a fragment, or derivative, or variant thereof in a sample from said subject and comparing said level, and/or said activity, or both said level and said activity, of at least one of said substances to a reference value representing a known disease or health status, thereby diagnosing or prognosticating said neurodegenerative disease in said subject, or determining the propensity or predisposition of said subject to develop a neurodegenerative disease.

In another aspect, the present invention provides a method of monitoring the progression of a neurodegenerative disease in a subject, comprising determining a level, or an activity, or both said level and said activity, of at least one substance which is selected frorn the group consisting of a transcription product of the SOAT1 gene, and/or a translation product of the SOAT1 gene, and/or of a fragment, or derivative, or variant thereof in a sample from said subject; and comparing said level, or said activity, or both said level and said activity, of at least one of said substances to a reference value representing a known disease or health status, thereby monitoring the progression of a neurodegenerative disease in said subject.

In a further aspect, the present invention provides a method of evaluating a treatment for a neurodegenerative disease, comprising determining a level, or an activity, or both said level and said activity, of at least one substance which is selected from the group consisting of a transcription product of the SOAT1 gene, and/or a translation product of the SOAT1 gene, and/or of a fragment, or derivative, or variant thereof in a sample obtained from a subject being treated for a neurodegenerative disease; and comparing said level, or said activity, or both said level and said activity, of at least one of said substances to a reference value representing a known disease or health status, thereby evaluating said treatment for a neurodegenerative disease.

In a preferred embodiment of the invention, the sample to be analyzed for determining a level, or an activity, or both said level and said activity, of at least one substance which is selected from the group consisting of a transcription product of the SOAT1 gene, and/or a translation product of the SOAT1 gene, and/or a fragment, or derivative, or variant thereof is taken from the group comprising body fluid, preferably cerebrospinal fluid, saliva, urine, mucus, blood, serum plasma, or a tissue, or cells like skin fibroblasts. Most preferably, the sample is taken from cerebrospinal fluid.

Preferably, the methods of diagnosis, prognosis, monitoring the progression or evaluating a treatment for a neurodegenerative disease, according to the instant invention, can be practiced ex corpore, and such methods preferably relate to samples, for instance, body fluids or cells, removed, collected, or isolated from a subject or patient.

In a preferred embodiment of the invention, the reference value of a level, or an activity, or both said level and said activity, of a transcription product of the SOAT1 gene, and/or a translation product of the SOAT1 gene, and/or a fragment, or derivative, or variant thereof is that in a sample from a subject not suffering from a neurodegenerative disease.

The determination of a level of transcription products of the SOAT1 gene can be performed in a sample from a subject using Northern blots with probes specific for said gene. Another preferred method of measuring said level is by quantitative PCR with primer combinations which amplify said gene-specific sequences from cDNA obtained by reverse transcription of RNA extracted from a sample of a subject. It might further be preferred to measure transcription products by means of chip-based microarray technologies. These techniques are known to those of ordinary skill in the art (see Sambrook and Russell, Molecular Cloning: A Laboratory Manual, Cold Spring Harbor Laboratory Press, Cold Spring Harbor, N.Y., 2001; Schena M., Microarray Biochip Technology, Eaton Publishing, Natick, Mass., 2000). An example of an immunoassay is the detection and measurement of enzyme activity as disclosed and described in the patent application WO 02/14543.

Furthermore, a level and/or an activity of a translation product of the SOAT1 gene (e.g. the enzyme ACAT1) and/or of a fragment, or derivative, or variant thereof, and/or a level of activity of said translation product of the SOAT1 gene, and/or of a fragment, or derivative, or variant thereof, can be detected using a Western blot analysis, an immunoassay, an enzyme activity assay, and/or a binding assay. These assays can measure the amount of binding between said translation product and an anti-polypeptide antibody by the use of enzymatic, chromodynamic, radioactive, magnetic, or luminescent labels which are attached to either the anti-polypeptide antibody or a secondary antibody which binds the anti-polypepfide antibody. In addition, other high affinity ligands may be used. Immunoassays which can be used include e.g. ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999 and Edwards R, Immunodiagnostics: A Practical Approach, Oxford University. Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000). Enzymatic activity of ACAT1 may be measured by in vitro, cell-based, or in vivo assays. Conveniently, ACAT1 enzymatic activity can, for instance, be determined using an acyltransferase activity assay.

In a preferred embodiment, the provided methods of diagnosing or prognosticating a neurodegenerative disease in a subject, or determining the propensity or predisposition of a subject to develop a neurodegenerative disease, or monitoring a treatment, or evaluating a treatment of a neurodegenerative disease further comprise comparing a level, or an activity, or both said level and said activity, of a transcription product of the SOAT1 gene, and/or a translation product of the SOAT1 gene, and/or a fragment, or derivative, or variant thereof in a series of samples taken from said subject over a period of time. In another preferred embodiment, said subject receives a treatment prior to one or more sample gatherings. It is a further preferred embodiment to determine said level, or said activity, or both said level and/or said activity, in said samples before and after said treatment of said subject.

In another aspect, the invention features a kit for diagnosing or prognosticating a neurodegenerative disease, in particular AD, in a subject, or determining the propensity or predisposition of a subject to develop a neurodegenerative disease, in particular AD, said kit comprising:
(a) at least one reagent which is selected from the group consisting of (i) reagents that selectively detect a transcription product of the SOAT1 gene, (ii) reagents that selectively detect a translation product of the SOAT1 gene, (iii) reagents that selectively detect the presence or absence of a variation in the SOAT1 gene; and
(b) an instruction for diagnosing, or prognosticating a neurodegenerative disease, in particular AD, or determining the propensity or predisposition of a subject to develop such a disease by (i) detecting a level, or an activity, or both said level and said activity, of said transcription product and/or said translation product of the SOAT1 gene, in a sample from said subject; and/or detecting the presence or absence of a variation in the SOAT1 gene in a sample from said subject; and (ii) diagnosing or prognosticating a neurodegenerative disease, in particular AD, or determining the propensity or predisposition of said subject to develop such a disease,
   wherein a varied level, or activity, or both said level and said activity, of said transcription product and/or said translation product compared to a reference value representing a known health status; or a level, or activity, or both said level and said activity, of said transcription product and/or said translation product similar or equal to a reference value representing a known disease status; or the presence of a variation in the SOAT1 gene indicates a diagnosis or prognosis of a neurodegenerative disease, in particular AD, or a measure for the propensity or predisposition of developing such a disease.

It is preferred that the reagents of the kit selectively detect the single nucleotide polymorphism located at chromosomal position 180457672, 29 bp downstream from the 3′-end of the ACAT1 mRNA (single nucleotide polymorphism identification number: rs1044925) in the SOAT1 gene. It is further preferred that the variation is a C to A transversion. It is further preferred, that for the purpose of diagnosing or prognosticating a neurodegenerative disease, in particular AD, in said subject, or determining the propensity or predispositon of said subject to develop such a disease, said subject carries both A-alleles.

It is further preferred that the reagents of the kit selectively detect either of all, or several of all, or all of the single nucleotide polymorphisms located within coding regions, intronic regions, within the 3′ UTR or the 3′ flanking region of the SOAT1 gene, said SNPs are in particular the SNPs rs3753526, rs3753527, rs1803033, rs4652366, rs2484437, rs2184575, rs2065761 and rs2065762.

The kit according to the present invention may be particularly useful for the identification of individuals that are at risk of developing a neurodegenerative disease, in particular AD. Consequently, the kit, according to the invention, may serve as a means for targeting identified individuals for early preventive measures or therapeutic intervention prior to disease onset, before irreversible damage in the course of the disease has been inflicted. Furthermore, in preferred embodiments, the kit featured in the invention is useful for monitoring a progression of a neurodegenerative disease, in particular AD, in a subject. It is further useful in monitoring success or failure of therapeutic treatment of said subject.

In another aspect, the invention features a method of treating or preventing Alzheimer's disease or related neurodegenerative diseases, in a subject comprising the administration to said subject in a therapeutically or prophylactically effective amount of an agent or agents which directly or indirectly affect a level, or an activity, or both said level and said activity, of (i) the SOAT1 gene, and/or (ii) a transcription product of the SOAT1 gene, and/or (iii) a translation product of the SOAT1 gene, and/or (iv) a fragment, or derivative, or variant of (i) to (iii). Said agent may comprise a small molecule, or it may also comprise a peptide, an oligopeptide, or a polypeptide. Said peptide, oligopeptide, or polypeptide may comprise an amino acid sequence of a translation product of the SOAT1 gene, or a fragment, or derivative, or a variant thereof. An agent for treating or preventing a neurodegenerative disease, in particular AD, according to the instant invention, may also consist of a nucleotide, an oligonucleotide, or a polynucleotide. Said oligonucleotide or polynucleotide may comprise a nucleotide sequence of the SOAT1 gene either in sense orientation or in antisense orientation.

In preferred embodiments, the method comprises the application of per se known methods of gene therapy and/or antisense nucleic acid technology to administer said agent or agents. In general, gene therapy comprises several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc Chem Res 1993, 26:274-278 and Mulligan, Science 1993, 260: 926-931) and include direct gene-transfer techniques such as mechanical microinjection of DNA into a cell as well as indirect techniques employing biological vectors (like recombinant viruses, especially retroviruses) or model liposomes, or techniques based on transfection with DNA coprecipitation with polycations, cell membrane pertubation by chemical (solvents, detergents, polymers, enzymes) or physical means (mechanic, osmotic, thermic, or electric shocks). The postnatal gene transfer into the central nervous system has been described in detail (see e.g. Wolff, Curr Opin Neurobiol 1993, 3:743-748).

In particular, the invention features a method of treating or preventing a neurodegenerative disease by means of antisense nucleic acid therapy, i.e. the down-regulation of an inappropriately expressed or defective gene by the introduction of antisense nucleic acids or derivatives thereof into certain critical cells (see e.g. Gillespie, DN&P 1992, 5:389-395; Agrawal and Akhtar, Trends Biotechnol 1995, 13:197-199; Crooke, Biotechnology 1992, 10:882-6). Apart from hybridization strategies, the application of ribozymes, i.e. RNA molecules that act as enzymes, destroying RNA that carries the message of disease has also been described (see e.g. Barinaga, Science 1993, 262:1512-1514). In preferred embodiments, the subject to be treated is a human, and therapeutic antisense nucleic acids or derivatives thereof are directed against the human SOAT1 gene. It is preferred that cells of the central nervous system, preferably the brain, of a subject are treated in such a way. Cell penetration can be performed by known strategies such as coupling of antisense nucleic acids and derivatives thereof to carrier particles, or the above described techniques. Strategies for administering targeted therapeutic oligodeoxynucleotides are known to those of skill in the art (see e.g. Wickstrom, Trends Biotechnol 1992, 10: 281-287). In some cases, delivery can be performed by mere topical application. Further approaches are directed to intracellular expression of antisense RNA. In this strategy, cells are transformed ex vivo with a recombinant gene that directs the synthesis of an RNA that is complementary to a region of target nucleic acid. Therapeutical use of intracellularly expressed antisense RNA is procedurally similar to gene therapy. A recently developed method of regulating the intracellular expression of genes by the use of double-stranded RNA, known variously as RNA interference (RNAi), can-be another effective approach for nucleic acid therapy (Hannon, Nature 2002, 418: 244-251).

In further preferred embodiments, the method comprises grafting donor cells into the central nervous system, preferably the brain, of said subject, or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection or liposomal mediated transfection (see Mc Celland and Pardee, Expression Genetics: Accelerated and High-Throughput Methods, Eaton Publishing, Natick, Mass., 1999).

In preferred embodiments, said agent for treating and preventing a neurodegenerative disease, in particular AD, is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector. Said agent, particularly a therapeutic protein, can further be administered to said subject by a process comprising the injection or the systemic administration of a fusion protein, said fusion protein consisting of a fusion of a protein transduction domain with said agent.

Methods of treatment, according to the present invention, comprise the application of therapeutic cloning, transplantation, and stem cell therapy using embryonic stem cells or embryonic germ cells and neuronal adult stem cells, combined with any of the previously described cell- and gene therapeutic methods. Stem cells may be totipotent or pluripotent. They may also be organ-specific. Strategies for repairing diseased and/or damaged brain cells or tissue comprise (i) taking donor cells from an adult tissue. Nuclei of those cells are transplanted into unfertilized egg cells from which the genetic material has been removed. Embryonic stem cells are isolated from the blastocyst stage of the cells which underwent somatic cell nuclear transfer. Use of differentiation factors then leads to a directed development of the stem cells to specialized cell types, preferably neuronal cells (Lanza et al., Nature Medicine 1999, 9: 975-977), or (ii) purifying adult stem cells, isolated from the central nervous system, or from bone marrow (mesenchymal stem cells), for in vitro expansion and subsequent grafting and transplantation, or (iii) directly inducing endogenous neural stem cells to proliferate, migrate, and differentiate into functional neurons (Peterson DA, Curr. Opin. Pharmacol. 2002, 2: 34-42). Adult neural stem cells are of great potential for repairing damaged or diseased brain tissues, as the germinal centers of the adult brain are free of neuronal damage or dysfunction (Colman A, Drug Discovery World 2001, 7: 66-71).

In preferred embodiments, the subject for treatment or prevention, according to the present invention, can be a human, an experimental animal, e.g. a mouse or a rat, a fish, a fly, or a worm; a domestic animal, or a non-human primate. The experimental animal can be an animal model for a neuro-degenerative disorder, e.g. a transgenic mouse and/or a knock-out mouse with an Alzheimer's-type neuropathology.

In a further aspect, the invention features a modulator of an activity, or a level, or both said activity and said level of at least one substance which is selected from the group consisting of (i) the SOAT1 gene and/or (ii) a transcription product of the SOAT1 gene, and/or (iii) a translation product of the SOAT1 gene, and/or (iv) a fragment, or derivative, or variant of (i) to (iii).

In an additional aspect, the invention features a pharmaceutical composition comprising said modulator and preferably a pharmaceutical carrier. Said carrier refers to a diluent, adjuvant, excipient, or vehicle with which the modulator is administered.

In a further aspect, the invention features a modulator of an activity, or a level, or both said activity and said level of at least one substance which is selected from the group consisting of (i) the SOAT1 gene, and/or (ii) a transcription product of the SOAT1 gene and/or (iii) a translation product of the SOAT1 gene, and/or (iv) a fragment, or derivative, or variant of (i) to (iii) for use in a pharmaceutical composition.

In another aspect, the invention provides for the use of a modulator of an activity, or a level, or both said activity and said level of at least one substance which is selected from the group consisting of (i) the SOAT1 gene, and/or (ii) a transcription product of the SOAT1 gene, and/or (iii) a translation product of the SOAT1 gene, and/or (iv) a fragment, or derivative, or variant of (i) to (iii) for a preparation of a medicament for treating or preventing a neurodegenerative disease, in particular Alzheimer's disease.

In one aspect, the present invention also provides a kit comprising one or more containers filled with a therapeutically or prophylactically effective amount of said pharmaceutical composition.

In a further aspect, the invention features a recombinant, non-human animal comprising a non-native gene sequence coding for a translation product of the SOAT1 gene, or a fragment, or a derivative, or a variant thereof. The generation of said recombinant, non-human animal comprises (i) providing a gene targeting construct containing said gene sequence and a selectable marker sequence, and (ii) introducing said targeting construct into a stem cell of a non-human animal, and (iii) introducing said non-human animal stem cell into a non-human embryo, and (iv) transplanting said embryo into a pseudopregnant non-human animal, and (v) allowing said embryo to develop to term, and (vi) identifying a genetically altered non-human animal whose genome comprises a modification of said gene sequence in both alleles, and (vii) breeding the genetically altered non-human animal of step (vi) to obtain a genetically altered non-human animal whose genome comprises a modification of said endogenous gene, wherein said gene is mis-expressed, or under-expressed, or over-expressed, and wherein said disruption or alteration results in said non-human animal exhibiting a predisposition to developing symptoms of neuropathology similar to a neurodegenerative disease, in particular Alzheimer's disease. Strategies and techniques for the generation and construction of such an animal are known to those of ordinary skill in the art (see e.g. Capecchi, Science 1989, 244:1288-1292 and Hogan et al., Manipulating the Mouse Embryo: A Laboratory Manual, Cold Spring Harbor Laboratory Press, Cold Spring Harbor, N.Y., 1994 and Jackson and Abbott, Mouse Genetics and Transgenics: A Practical Approach, Oxford University Press, Oxford, England, 1999). It is preferred to make use of such a recombinant non-human animal as an animal model for investigating neurodegenerative diseases, in particular Alzheimer's disease. Such an animal may be useful for screening, testing and validating compounds, agents and modulators in the development of diagnostics and therapeutics to treat neurodegenerative diseases, in particular Alzheimer's disease.

In another aspect, the invention features an assay for screening for a modulator of neurodegenerative diseases, in particular Alzheimer's disease, or related diseases and disorders of one or more substances selected from the group consisting of (i) the SOAT1 gene, and/or (ii) a transcription product of the SOAT1 gene, and/or (iii) a translation product of the SOAT1 gene, and/or (iv) a fragment, or derivative, or variant of (i) to (iii). This screening method comprises (a) contacting a cell with a test compound, and (b) measuring the level, or the activity, or both the level and the activity of one or more substances recited in (i) to (iv), and (c) measuring the level, or the activity, or both the level and the activity of said substances in a control cell not contacted with said test compound, and (d) comparing the levels of the substance in the cells of step (b) and (c), wherein an alteration in the level and/or activity of said substances in the contacted cells indicates that the test compound is a modulator of said diseases and disorders.

In one further aspect, the invention features a screening assay for a modulator of neurodegenerative diseases, in particular Alzheimer's disease, or related diseases and disorders of one or more substances selected from the group consisting of (i) the SOAT1 gene, and/or (ii) a transcription product of the SOAT1 gene, and/or (iii) a translation product of the SOAT1 gene, and/or (iv) a fragment, or derivative, or variant of (i) to (iii), comprising (a) administering a test compound to a test animal which is predisposed to developing or has already developed symptoms of a neurodegenerative disease or related diseases or disorders, and (b) measuring the level and/or activity of one or more substances recited in (i) to (iv), and (c) measuring the level and/or activity of said substances in a matched control animal which is equally predisposed to developing or has already developed said symptoms of said diseases and to which animal no such test compound has been administered, and (d) comparing the level and/or activity of the substance in the animals of step (b) and (c), wherein an alteration in the level and/or activity of substances in the test animal indicates that the test compound is a modulator of said diseases and disorders.

In a preferred embodiment, said test animal and/or said control animal is a recombinant, non-human animal which expresses the SOAT1 gene, or a fragment, or a derivative, or a variant thereof, under the control of a transcriptional regulatory element which is not the native SOAT1 gene transcriptional control regulatory element.

In another embodiment, the present invention provides a method for producing a medicament comprising the steps of (i) identifying a modulator of neurodegenerative diseases by a method of the herein aforementioned screening assays and (ii) admixing the modulator with a pharmaceutical carrier. However, said modulator may also be identifiable by other types of screening assays.

In another aspect, the present invention provides for a method of testing a compound, preferably an assay for screening a plurality of compounds, for inhibition of binding between a ligand and a SOAT1 gene product, or a fragment, or derivative, or variant thereof. Said method comprises the steps of (i) adding a liquid suspension of said SOAT1 gene product, or a fragment, or a derivative, or a variant thereof, to a plurality of containers, and (ii) adding a compound, preferably a plurality of compounds, to be screened for said inhibition to said plurality of containers, and (iii) adding a detectable ligand, preferably a fluorescently detectable ligand, to said containers, and (iv) incubating the liquid suspension of said SOAT1 gene product, or said fragment, or derivative, or variant thereof, and said compounds, and said detectable ligand, preferably said fluorescently detectable ligand, and (v) measuring the amounts of detectable ligand, preferably of fluorescence associated with said SOAT1 gene product, or with said fragment, or derivative, or variant thereof, and (vi) determining the degree of inhibition by one or more of said compounds of binding of said ligand to said SOAT1 gene product, or said fragment, or derivative, or variant thereof. Instead of utilizing a fluorescently detectable label, it might in some aspects be preferred to use any other detectable label known to the person skilled in the art, e.g. radioactive label, and detect it accordingly. Said method may be useful for the identification of novel compounds as well as for evaluating compounds which have been improved or otherwise optimized in their ability to inhibit the binding of a ligand to a SOAT1 gene product, or a fragment, or derivative, or variant thereof. One example of a fluorescent binding assay, in this case based on the use of carrier particles, is disclosed and described in patent application WO 00/52451. A further example is the competitive assay method as described in patent WO 02/01226. Preferred signal detection methods for screening assays of the instant invention are described in the following patent applications: WO 96/13744, WO 98/16814, WO 98/23942, WO 99/17086, WO 99/34195, WO 00/66985, WO 01/59436, WO 01/59416.

In one further embodiment, the present invention provides a method for producing a medicament comprising the steps of (i) identifying a compound as an inhibitor of binding between a ligand and a SOAT1 gene product by the herein aforementioned inhibitory binding assay and (ii) admixing the compound with a pharmaceutical carrier. However, said compound may also be identifiable by other types of screening assays.

In one further aspect, the invention features a method of testing a compound, preferably an assay for screening a plurality of compounds, to determine the degree of binding of said compound or compounds to a SOAT1 gene product, or to a fragment ,or derivative, or variant thereof. Said method comprises the steps of (i) adding a liquid suspension of said SOAT1 gene product, or a fragment, or derivative, or variant thereof, to a plurality of containers, and (ii) adding a detectable compound, preferably a plurality of detectable compounds, in particular fluorescently detectable compounds, to be screened for said binding to said plurality of containers, and (iii) incubating the liquid suspension of said SOAT1 gene product, or said fragment, or derivative, or variant thereof, and said detectable, preferably said fluorescently detectable compound, preferably said plurality of detectable compounds, and (iv) measuring the amounts of detectable compound or fluorescence associated with said SOAT1 gene product, or with said fragment, or derivative, or variant thereof, and (v) determining the degree of binding by one or more of said compounds to said SOAT1 gene product, or said fragment, or derivative, or variant thereof. In this type of assay it might be preferred to use a fluorescent label. However, any other type of detectable label might also be employed. Said method may be useful for the identification of novel compounds as well as for evaluating compounds which have been improved or otherwise optimized in their ability to bind to a SOAT1 gene product, or a fragment, or a derivative, or a variant thereof.

In one further embodiment, the present invention provides a method for producing a medicament comprising the steps of (i) identifying a compound as a binder to a SOAT1 gene product by the herein aforementioned binding assays and (ii) admixing the compound with a pharmaceutical carrier. However, said compound may also be identifiable by other types of screening assays.

In another embodiment, the present invention provides for a medicament obtainable by any of the methods according to the herein claimed screening assays. In one further embodiment, the instant invention provides for a medicament obtained by any of the methods according to the herein claimed screening assays.

The present invention features a protein molecule, said protein molecule being a translation product of the SOAT1 gene, or a fragment, or derivative, or variant thereof, for the use as a diagnostic target for detecting a neurodegenerative disease, preferably Alzheimer's disease.

The present invention further features a protein molecule, said protein molecule being a translation product of the SOAT1 gene, or a fragment, or derivative, or variant thererof, for the use as a screening target for reagents or compounds preventing, or treating, or ameliorating a neurodegenerative disease, preferably Alzheimer's disease.

The present invention features an antibody which is specifically immunoreactive with an immunogen, wherein said immunogen is a translation product of the SOAT1 gene or a fragment, or derivative or varaint thereof. The immunogen may comprise immunogenic or antigenic epitopes of portions of a translation product of said genes, wherein said immunogenic or antigenic portion of a translation product is a polypeptide, and wherein said polypeptide elicits an antibody response in an animal, and wherein said polypeptide is immunospecifically bound by said antibody. Methods for generating antibodies are well known in the art (see Harlow et al., Antibodies, A Laboratory Manual, Cold Spring Harbor Laboratory Press, Cold Spring Harbor, N.Y., 1988). The term “antibody”, as employed in the present invention, encompasses all forms of antibodies known in the art, such as polyclonal, monoclonal, chimeric, recombinatorial, anti-idiotypic, humanized, or single chain antibodies as well as fragments thereof (see Dubel and Breitling, Recombinant Antibodies, Wiley-Liss, New York, N.Y., 1999). Antibodies of the present invention are useful, for instance, in a variety of diagnostic and therapeutic methods, based on state-of-the-art techniques (see Harlow and Lane, Using Antibodies: A Laboratory Manual, Cold Spring Harbor Laboratory Press, Cold Spring Harbor, N.Y., 1999 and Edwards R., Immunodiagnostics: A Practical Approach, Oxford University Press, Oxford, England, 1999) such as enzyme-immuno assays (e.g. enzyme-linked immunosorbent assay, ELISA), radioimmuno assays, chemoluminescence-immuno assays, Western-blot, immunoprecipitation and antibody microarrays. These methods involve the detection of translation products of the SOAT1 gene, or fragments, or derivatives, or variants thereof.

In a preferred embodiment of the present invention, said antibodies can be used for detecting the pathological state of a cell in a sample from a subject, comprising immunocytochemical staining of said cell with said antibody, wherein an altered degree of staining, or an altered staining pattern in said cell compared to a cell representing a known health status indicates a pathological state of said cell. Preferably, the pathological state relates to a neurodegenerative disease, in particular to Alzheimer's disease. Immuno-cytochemical staining of a cell can be carried out by a number of different experimental methods well known in the art. It might be preferred, however, to apply an automated method for the detection of antibody binding, wherein the determination of the degree of staining of a cell, or the determination of the cellular or subcellular staining pattern of a cell, or the topological distribution of an antigen on the cell surface or among organelles and other subcellular structures within the cell, are carried out according to the methods described in the U.S. Pat. No. 6,150,173.

Other features and advantages of the invention will be apparent from the following detailed description of the figures and examples which are illustrative only and not intended to limit the remainder of the disclosure in any way.

Table 1 shows SOAT1 genotype and allele distribution in control subjects and Alzheimer's disease patients.

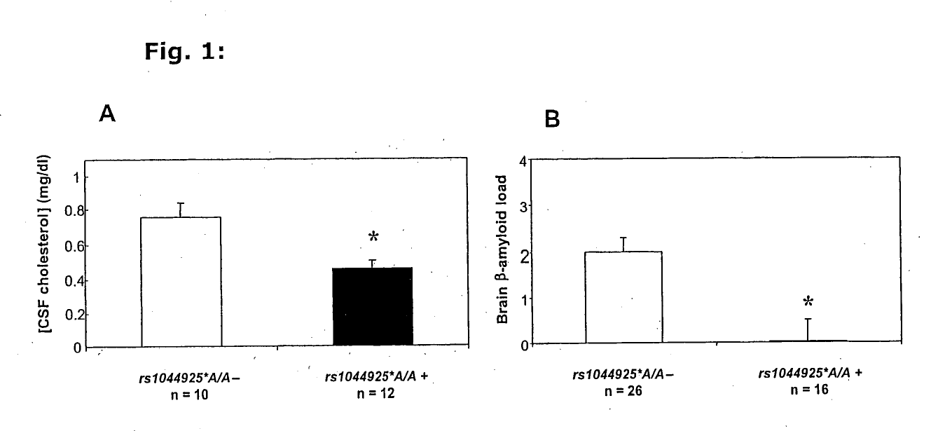

FIG. 1:

a) Decreased CSF cholesterol levels in A/A carriers (rs1044925 A/A+) in comparison to non-carriers (rs1044925 A/A−).

b) Decreased brain amyloid load in A/A carriers (rs1044925 A/A+) in comparison to non-carriers (rs1044925 A/A −).

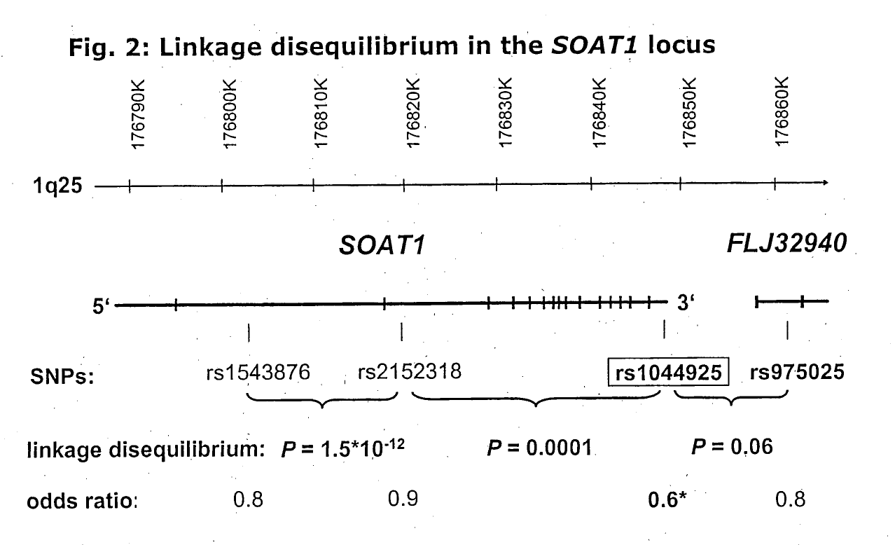

FIG. 2 schematically displays the region around the SOAT1 gene on chromosome 1q25 with the cDNA FLJ32940 (GenBank accession number AK057502) being located 3′ downstream of SOAT1 . A series of single nucleotide polymorphisms (SNPs) (i.e. SNP cluster ID rs1543876, rs2152318, rs1044925, rs975025) are indicated lying within or in the close proximity to the SOAT1 gene locus. At the bottom of the display the P-values indicating the degree of linkage disequilibrium and the odds ratios are shown.

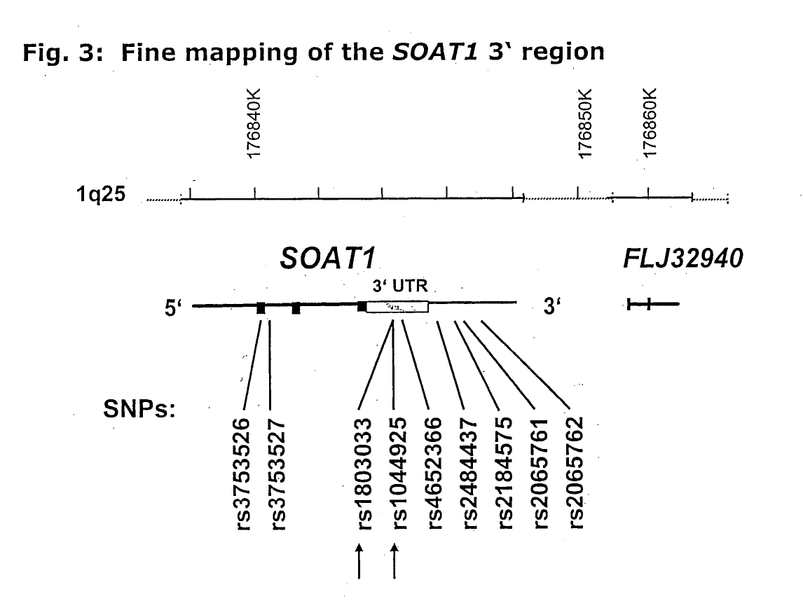

FIG. 3 illustrates the fine mapping of the 3′ region of the SOAT1 gene, represented by a thick bar, and the 3′ flanking region, indicated by the thin bar, respectively, harboring several single nucleotide polymorphisms. The single nucleotide polymorphism (SNP) rs3753526 for instance, is located within a coding region of the SOAT1 gene (vertical black bars). The SNPs rs1803033 and rs1044925 are marked by an arrow, they are located within the 3′ untranslated region (3′ UTR, open rectangle) of the SOAT1 gene.

### EXAMPLE

To determine whether polymorphisms in the SOAT1 gene may be associated with an altered risk for AD, we conducted, in two ethnically and geographically distinct populations a genetic association study in 325 patients diagnosed with AD according to the NINCDS-ADRDA criteria (McKhann et al., Neurology 1984, 34:939-44) and 359 healthy control subjects (HCS). The participants were genotyped for two informative single nucleotide polymorphisms (SNPs) in the 5′ (SNP cluster ID rs2152318). and the 3′ (SNP cluster ID rs1044925) region of SOAT1 (see FIG. 2) using the Masscode system (Kokoris et al., Mol Diagn 2000, 5:329-40). In addition, the polymorphic site SNP ID rs975025 which is located at the 5′ end of the cDNA sequence FLJ32940 of a hitherto hypothetical protein (Genbank accession number AK057502) was genotyped in a coillective of 801 patients diagnosed with AD and 88 healthy control subjects. The locus of the cDNA sequence FLJ32940 is in close proximity 3′-downstream of the SOAT1 gene sequence. Informations on polymorphic sites of SOAT1 and of FLJ32940 were derived from the data base of single nucleotide polymorphisms (dbSNP) established by the National Center for Biotechnology Information (www. ncbi. nim. nih. gov/SNP/index. html). Genotype and allele frequencies between AD and HCS groups were compared by Pearson's χ² tests. The A/A genotype of SNP rs1044925 was significantly under-represented in AD patients (31.7% AD vs. 42.9 HCS, p=0.002) (Table 1) and was associated with reduced risk for AD (odds ratio=0.6, 95% confidence interval=0.5-0.8) Separate analysis in the two populations yielded similar results. SNP rs2152318, lying within the 5+ region of SOAT1, was not associated with AD (p=0.9). Both SNPs were in incomplete linkage disequilibrium as estimated by the EH program (see FIG. 2) (Terwilliger and Ott, Handbook of Human Genetic Linkage, Baltimore: The Johns Hopkins University Press, p. 189-198, 1994). The two-locus haplotype spanning both SNPs was not associated with AD (p=0.155) indicating tight linkage of the functionally relevant polymorphism with SNP rs1044925. Furthermore, the SNP rs975025, lying in the gene coding for cDNA FLJ32940, was not associated with AD (p=0.2), whereas the analysis of the SNP rs1044925 using the same group of 80 AD patients and 88 healthy control subjects as for the examination of SNP rs975025 revealed a result already indicating association with AD (p<0.05). Since SNP rs975025 (cDNA FLJ32940) shows no association with AD, and since there is only a weak, non-significant linkage of said SNP and SNP rs1044925 (SOAT1 gene), there is evidence for the notion that the SOAT1 gene polymorphisms, i.e. the SNPs rs3753526, rs3753527, rs1803033, rs1044925, rs4652366, rs2484437, rs2184575, rs2065761 and rs2065762, are associated with an altered risk for AD and, thus, are useful as diagnostic markers for AD.

To assess if SNP rs1044925 is also associated with central nervous system cholesterol homeostasis, we measured, by combined gas chromatography/mass spectrometry (Dzeletovic et al., Anal Biochem 1995, 225:73-80), cholesterol as well as its precursors lathosterol and cholestanol and its catabolic derivative 24S-hydroxycholesterol in cerebrospinal fluid (CSF) of 22 healthy elderly. We found significantly (p=0.005) lower levels of cholesterol in CSF of A/A carriers than in non-carriers (FIG. 1a). Neither lathosterol nor cholestanol nor 24S-hydroxycholesterol were associated with the SNP rs1044925 A/A genotype (p>0.2). These results indicate that neither cholesterol synthesis nor cholesterol catabolism account for the observed difference of CSF cholesterol concentration. In line with the reported role of the enzyme ACAT1, this difference rather reflects genotype-specific changes in cholesterol distribution and compartmentation.

Because Aβ production depends on cholesterol distribution and on the function of ACAT1, we tested whether the SNP rs1044925 A/A genotype is also associated with brain β-amyloid load in humans. We assessed, by immunohistochemistry, the β-amyloid load in the medial temporal lobes of 42 elderly non-demented subjects, and staged them according to the phases described by Thal et al. (J Neuropathol Exp Neurol 2000, 59:733-48). The protective A/A genotype was specifically associated with low brain amyloid load (p=0.033, FIG. 1b) and had no impact on Braak staging (p=0.393). On the background of disturbed cholesterol metabolism, ACAT1 deficient mice have low circulating cholesterol levels (Accad M., J Clin Invest 2000, 105:711-719). The disclosure in the present invention of low CSF cholesterol levels in SNP rs1044925 A/A carriers suggests that this genotype is associated with a loss of function of ACAT1 and, as a consequence thereof, beneficial for the prevention of neurodegenerative diseases, in particular Alzheimer's disease.

1. A method for diagnosing or prognosticating a neurodegenerative disease in a subject, or determining the propensity or predisposition of a subject to develop a neurodegenerative disease, comprising detecting in a sample obtained from said subject the presence or absence of a variation in the gene coding for “acyl-coenzyme A: cholesterol acyltransferase 1”, wherein the presence of a variation in the gene coding for “acyl-coenzyme A: cholesterol acyltransferase 1” in said subject indicates a diagnosis or prognosis of a neurodegenerative disease, or a measure for the propensity or predisposition to develop such a disease as compared to a subject who does not carry a variation in said gene.

2. The method according to claim 1 wherein said variation in the gene coding for “acyl-coenzyme A: cholesterol acyltransferase 1” is a single nucleotide polymorphism located at chromosomal position 180457672 (single nucleotide polymorphism identification number: rs1044925).

3. The method according to claim 1 wherein said variation is a C to A transversion.

4. The method according to claim 1 wherein said subject is homozygous in respect to said variation.

5. The method according to claim 1 wherein said neurodegenerative disease is Alzheimer's disease.

6. A method for diagnosing or prognosticating a neurodegenerative disease, in particular Alzheimer's disease, in a subject, or determining the propensity or predisposition of a subject to develop such a disease, comprising:
determining a level, or an activity, or both said level and said activity, of at least one substance which is selected from the group consisting of a transcription product of the gene coding for “acyl-coenzyme A: cholesterol acyltransferase 1”, or a translation product of the gene coding for “acyl-coenzyme A: cholesterol acyltransferase 1”, or a fragment, or derivative, or variant thereof in a sample from said subject; and comparing said level, or said activity, or both said level and said activity, of at least one of said substances to a reference value representing a known disease or health status, thereby diagnosing or prognosticating a neurodegenerative disease, in particular Alzheimer's disease, in said subject, or determining the propensity or predisposition of said subject to develop such a disease.

7. A kit for diagnosing or prognosticating a neurodegenerative disease, in particular Alzheimer's disease, in a subject, or determining the propensity or predisposition of a subject to develop such a disease by:
(i) detecting a level, or an activity, or both said level and said activity, of said transcription product and/or said translation product of the gene coding for “acyl-coenzyme A: cholesterol acyltransferase 1”, in a sample obtained from said subject; and/or detecting the presence or absence of a variation in the gene coding for “acyl-coenzyme A: cholesterol acyltransferase 1” in a sample from said subject and (ii) comparing to a reference value representing a known health status, the level or activity, or both said level and said activity, of said transcription product and/or said translation product of the gene coding for “acyl-coenzyme A: cholesterol acyltransferase 1” and/or the presence of a variation in the gene coding for “acyl-coenzyme A: cholesterol acyltransferase 1”,
and said kit comprising:
at least one reagent which is selected from the group consisting of (i) reagents that selectively detect a transcription product of the gene coding for “acyl-coenzyme A: cholesterol acyltransferase 1”, (ii) reagents that selectively detect a translation product of the gene coding for “acyl-coenzyme A: cholesterol acyltransferase 1”, (iii) reagents that selectively detect the presence or absence of a variation in the gene coding for “acyl-coenzyme A: cholesterol acyltransferase 1”.

8. The kit according to claim 7 wherein said variation in the gene coding for “acyl-coenzyme A: cholesterol acyltransferase 1” is a single nucleotide polymorphism located at chromosomal position 180457672 (single nucleotide polymorphism identification number: rs2044925).
